(19) United States
(12) Patent Application Publication
(10) Pub. No.: US 2010/0298824 A1
(43) Pub. Date: Nov. 25, 2010

(54) Bipolar Ablation Device, System and Method for Minimally Invasive Isolation of Pulmonary Veins in a Sub-Xiphoid Approach

(75) Inventors: Paul T. Rothstein, Elk River, MN (US); Alison Lutterman, Minneapolis, MN (US); David Kim, Maple Grove, MN (US); Tom P. Daigle, Corcoran, MN (US)

Correspondence Address: Medtronic CardioVascular, Mounds View Facility South, 8200 Coral Sea Street N.E., Mounds View, MN, 55112, US

(73) Assignee: Medtronic, Inc.

(21) Appl. No.: 12/754,722

(22) Filed: Apr. 6, 2010

Related U.S. Application Data
(60) Provisional application No. 61/166,972, filed on Apr. 6, 2009.

(51) Int. Cl. A61B 18/14

(52) U.S. Cl. 606/41

(57) ABSTRACT

Structure and method for using a sub-xiphoid ablation clamp for ablating tissue of a patient. The clamp has an elongate shaft having a major axis, first and second opposing jaws configured to open and close along a first plane, a first and second ablation element positioned along the first and second jaws configured to ablate the tissue positioned therebetween, an actuable joint operatively coupled between the shaft and the opposing jaws and configured to move the opposing jaws to a selectable angle relative to the major axis of the elongate shaft along a second plane orthogonal to the first plane. The ablation clamp has a handle operatively coupled to the shaft having an actuator configured to actuate the actuable joint and a trigger mechanism to open and close the opposing jaws.

### PRIORITY

This application claims the benefit of U.S. Provisional Application No. 61/166,972, filed on Apr. 6, 2009, entitled “Bipolar Ablation Device, System and Method for Minimally Invasive Isolation of Pulmonary Veins in a Sub-Xiphoid Approach.”

### FIELD

The present invention is related to apparatus and methods for the ablation of tissue and, in particular, ablation of heart tissue.

### BACKGROUND

Atrial fibrillation is a common cardiac condition in which irregular heartbeats cause a decrease in the efficiency of the heart, sometimes due to variances in the electrical conduction system of the heart. In some circumstances, atrial fibrillation poses no immediate threat to the health of the individual suffering from the condition, but may, over time, result in conditions adverse to the health of the patient, including heart failure and stroke. But the case of many of individuals suffering from atrial fibrillation, symptoms affecting the patient's quality of life may occur immediately with the onset of the condition, including lack of energy, fainting and heart palpitations.

In some circumstances, atrial fibrillation may be treated through the application of defibrillation shocks. In cases of persistent atrial fibrillation, however, surgery may be required. A surgical procedure sometimes used for this condition is the ablation and isolation of tissue which may be responsible for the improper electrical conduction that causes atrial fibrillation. One such location of tissue which may be responsible for improper electrical conduction is at the junction of the pulmonary veins with the left atrium where spontaneous triggers for initiation of atrial fibrillation have been found. Patients who suffer from a paroxysmal form of atrial fibrillation experience short, self terminating episodes of atrial fibrillation. “Lone” atrial fibrillation occurs in patients who have either few or no other significant cardiac diseases.

In the past, direct access to the heart has been created by moving patient anatomy such as the ribcage out of the way. Such methods tend to create serious trauma to the patient. Access to the left pulmonary veins by an inferior approach to the heart may be relatively free from interference. However, ablation around the right pulmonary veins may be relatively more complicated due to the presence of the superior and inferior vena cava. In particular, while a sub-xiphoid approach to the heart, also known as a substernal approach to the heart, may be generally less traumatic to the patient than the direct approach, the presence of the inferior vena cava, in particular, may make a sub-xiphoid approach to the right pulmonary veins difficult or impossible.

### SUMMARY

An ablation clamp has been developed which allows sub-xiphoid access to the right pulmonary veins. The ablation clamp is provided with an actuable joint and a means to actuate the actuable joint. When the ablation clamp is inserted into the patient on a sub-xiphoid approach the jaws of the clamp may be maneuvered around the inferior vena cava. Then, when past the inferior vena cava, the actuable joint may be actuated to swing the jaws of the ablation clamp around into proximity of the right pulmonary veins. After the right pulmonary veins are ablated the ablation clamp may be returned to its unarticulated state and withdrawn. In this way, the right pulmonary veins may be ablated with reduced trauma to the patient.

Various embodiments of the ablation clamp utilize differing actuable joints. One embodiment utilizes a “gooseneck” joint. In the gooseneck joint, articulated segments provide flexibility. In an alternative embodiment a pivot joint on a pivot knuckle provides flexibility. In both embodiments, actuation of the actuable joint may be provided by an actuator on the ablation clamp which is easily accessible to a user.

In an embodiment, a sub-xiphoid ablation clamp for ablating tissue of a patient has an elongate shaft having a major axis, a proximal end and a distal end, first and second opposing jaws configured to open and close along a first plane, the first and second opposing jaws having a first and second ablation element positioned along the first and second jaws, respectively, configured to ablate the tissue positioned therebetween, an actuable joint operatively coupled between the distal end of the elongate shaft and the first and second opposing jaws, the actuable joint being configured to move the opposing jaws to a selectable angle relative to the major axis of the elongate shaft along a second plane orthogonal to the first plane of the opposing jaws and a handle operatively coupled to the proximal end of the elongate shaft. The handle has an actuator operatively coupled to the actuable joint and configured to actuate the actuable joint and a trigger mechanism operatively coupled to the first and second opposing jaws, the trigger mechanism being operable to open and close the opposing jaws.

In an embodiment, the actuable joint is comprised of a plurality of articulated segments.

In an embodiment, the actuable joint is a gooseneck.

In an embodiment, the actuable joint is configured to move the operable jaws along the second plane with respect to the major axis of the shaft only in a first direction.

In an embodiment, the actuable joint comprises a pivot joint.

In an embodiment, the actuator is configured to actuate the actuable joint to a plurality of predetermined angles relative to the major axis of the shaft.

In an embodiment, the plurality of predetermined angles are at predetermined increments.

In an embodiment, the predetermined increments are approximately ten degrees.

In an embodiment, a method of sub-xiphoid ablation of a vein of a heart of a patient uses a sub-xiphoid ablation clamp having an elongate shaft having a major axis, first and second opposing jaws configured to open and close along a first plane, the first and second opposing jaws comprising a first and second ablation element, an actuable joint operatively being configured to move the opposing jaws to a selectable angle relative to the major axis of the elongate shaft along a second plane orthogonal to the first plane of the opposing jaws. The method comprises inserting the ablation clamp within the patient from a sub-xiphoid direction, positioning the opposing jaws proximate the vein, moving the opposing jaws along the second plane to a particular selectable angle with respect to the major axis of the shaft position the vein between the opposing jaws, clamping the vein between the opposing jaws by closing the opposing jaws along the first plane, and delivering ablation energy to the vein from the first and second opposing electrodes.

In an embodiment, the vein is a right pulmonary vein.

In an embodiment, the method further has the step, before the inserting step, of creating an incision in skin of the patient below a sternum of the patient, and wherein the inserting step comprising inserting the sub-xiphoid ablation clamp into the incision.

In an embodiment, the method further has the step, after the creating an incision step, of creating an incision in a pericardium of the heart of the patient, creating a gap in the subxiphoid process, and passing the jaws of the sub-xiphoid ablation clamp though the incision in the pericardium and the gap in the subxiphoid process.

In an embodiment, the gap in the subxiphoid process is created by removing a portion of the subxiphoid process proximate a sternum of the patient.

### FIGURES

FIG. 1 is a view of a posterior aspect of a pericardial sac of a human heart with arteries and veins sectioned off;

FIGS. 2a and 2b are views of an ablation clamp;

FIG. 3 is a close-up view of a gooseneck joint;

FIG. 4 is an image of the ablation clamp of FIGS. 2a and 2b with the gooseneck articulated;

FIG. 5 is a view of ablation clamps of FIGS. 2a and 2b being used to ablate veins of the human heart;

FIG. 6 is a cutaway drawing of the ablation clamp of FIGS. 2a and 2b in use in a patient;

FIG. 7 is an image of an alternative ablation clamp using a pivot joint;

FIG. 8 is an image of the ablation clamp of FIG. 7 articulated with closed jaws;

FIG. 9 is a flowchart of using the ablation clamp of FIGS. 2a and 2b; and

FIG. 10 is a flowchart of inserting the ablation clamp of FIGS. 2a and 2b within a patient.

### DESCRIPTION

The entire content of U.S. Provisional Application Ser. No. 61/166,972, filed Apr. 6, 2009, is hereby incorporated by reference in its entirety.

Devices and methods disclosed herein are designed for isolation of the pulmonary veins in a minimally invasive environment. The sub-xiphoid region may be desirable because it is soft tissue, while a sub-xiphoid approach is relatively minimally invasive approach involving less trauma than a sternotomy. The ability to articulate the clamping mechanism allows the jaw mechanism to be more easily placed about the target tissue than with a wholly or generally rigid ablation device. A gooseneck design has an articulating neck that allows wires and tubing to be easily passed through.

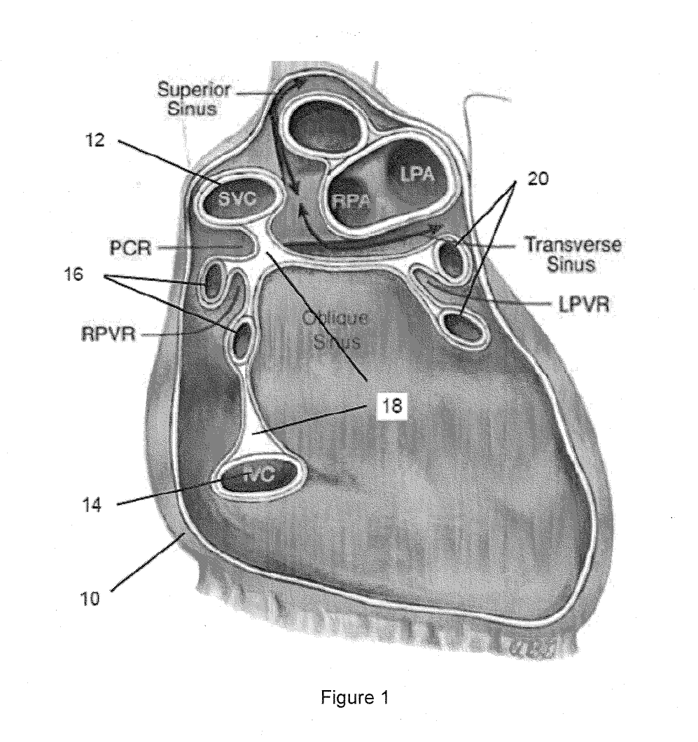

FIG. 1 shows a posterior view of a diagram of human heart 10.

Superior vena cava 12 and inferior vena cava 14 deliver de-oxygenated blood to the heart from the upper and lower regions of the body, respectively. The two right pulmonary veins 16 and the two left pulmonary veins 20 deliver oxygenated blood from the lungs to the left atrium. Pericardial reflections 18 extend between superior vena cava 12, inferior vena cava 14, right pulmonary veins 16 and left pulmonary veins 20.

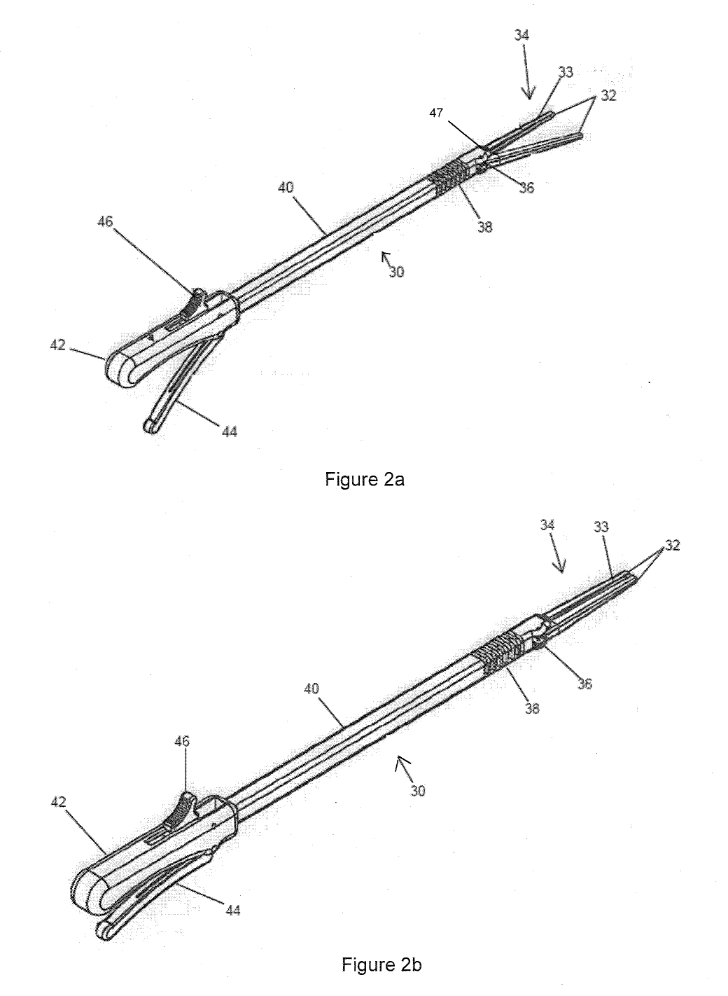

FIGS. 2a and 2b show a bipolar ablation device 30 configured to isolate pulmonary veins 16, 20 sing a sub-xiphoid approach. Bipolar ablation device 30 has linear opposing jaws 32 at its distal end 34 that close about pivot point 36. Ablation elements 33 are positioned along jaws 32 and are configured to ablate tissue around which jaws 32 are positioned. Jaws 32 are attached to an actuable joint (gooseneck) 38 that transitions into rigid, elongate shaft 40. Attached to shaft 40 at proximal end 42 of ablation device 30, is handle 42 having trigger mechanism 44 and actuator thumb slide 46. Gooseneck 38 may be used to enable insertion and to obtain proper orientation of jaws 32 with respect to pulmonary veins 16, 20.

In various embodiments, ablation device 30 is from approximately twelve (12) inches (30.5 centimeters) to approximately twenty (20) inches (50.8 centimeters) long from the tip of jaws 32 to the end of handle 42. In an embodiment, ablation device 30 is approximately sixteen (16) inches (40.6 centimeters) long. In such an embodiment, a combined length of jaws 32 and shaft 40 is approximately twelve (12) inches (30.5 centimeters). In alternative embodiments, the combined length of jaws 32 and shaft 40 may vary from approximately eight (8) inches (20.3 centimeters) to sixteen (16) inches (40.6 centimeters).

In various embodiments, jaws 32 are from approximately two (2.0) to four and one-half (4.5) inches (5.1 to 11.4 centimeters) in length. In an embodiment, jaws 32 are approximately four (4) inches (10.2 centimeters) long from the tips of jaws 32 to pivot 36. In such an embodiment, jaws 32 are approximately 3.3 inches (8.4 centimeters) long from the farthest extent 47 of shaft 40 to the tips of jaws 32. In such an embodiment, bi-bipolar electrodes 33 are approximately 3.2 inches (8.1 centimeters) long and approximately 0.12 inches (0.3) centimeters) wide. In alternative embodiments, electrodes 33 range from approximately 1.9 inches (4.8 centimeters) long to 4.0 inches (10.2 centimeters) long.

In certain embodiments, shaft 40 is a shaft of varying cross-sections, including square cross-sections and circular cross-sections, the various cross-sections being of varying dimensions. As depicted in FIG. 2a, shaft 40 has a square cross-section 0.5 inches (1.3 centimeters) on each side. In alternative embodiments utilizing a square cross-section, shaft 40 may range from 0.2 inches (0.5 centimeters) to 1.0 inches (2.5 centimeters). In the embodiment of FIG. 2a, shaft 40 from handle 42 to farthest extent 47 of shaft 40 is approximately 8.7 inches (22.1 centimeters) long, with shaft 40 from farthest extent 47 to gooseneck 38 being approximately 1.2 inches (3.0 centimeters) long.

As depicted in FIG. 2a, jaws 32 are open and trigger 44 is not compressed. In various embodiments, jaws 32 open at an angle of between 20.0 degrees and 45.0 degrees. In an embodiment, jaws 32 open at an angle of approximately 35.0 degrees. As depicted in FIG. 2b, jaws 32 are closed. As illustrated, trigger 44 is compressed, thereby closing jaws 32 along a plane, the plane extending along a plane encompassing jaws 32 when jaws 32 are open. In an embodiment, compressing trigger 44 so that jaws 32 are closed is a first stage, e.g., first detent, in trigger 44, with a second stage, e.g., further compressing trigger 44 to a second detent, causing the delivery of ablation energy to ablation elements 33. In an embodiment, the second stage may be implemented only after the completion of the first stage, i.e., jaws 32 are compressed. In alternative embodiments, additional triggers may deliver ablation energy to ablation elements 33, or trigger 44 may cause the delivery of ablation energy at alternative stages in the trigger mechanism or without actuation of trigger 44.

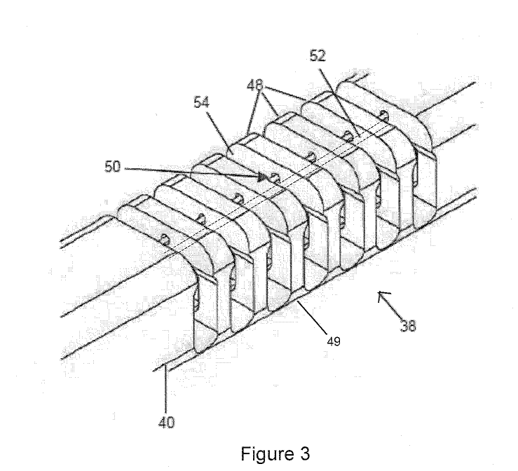

FIG. 3 shows a close view of gooseneck 38. Shaft segments 48 are coupled at bottom portion 49 of gooseneck 38. Cable 50 is connected to thumb slide 46 and to a distal end of gooseneck 38. In alternative embodiments, articulating mechanisms other than thumb slide 46 may be utilized which are well known in the art. Cable 50 runs through shaft 40. In an embodiment, additional wires 52 (obscured) may run through gooseneck 38 and shaft 40, e.g., parallel with cable 50, to provide electrical connectivity between ablation elements 33 and other electronic devices positioned on jaws 32 and peripheral devices which provide energy and receive data from ablation elements 33 and other electronic components, as appropriate. In various embodiments, wires 52 may be connected to connection ports and jacks to interface with peripheral devices.

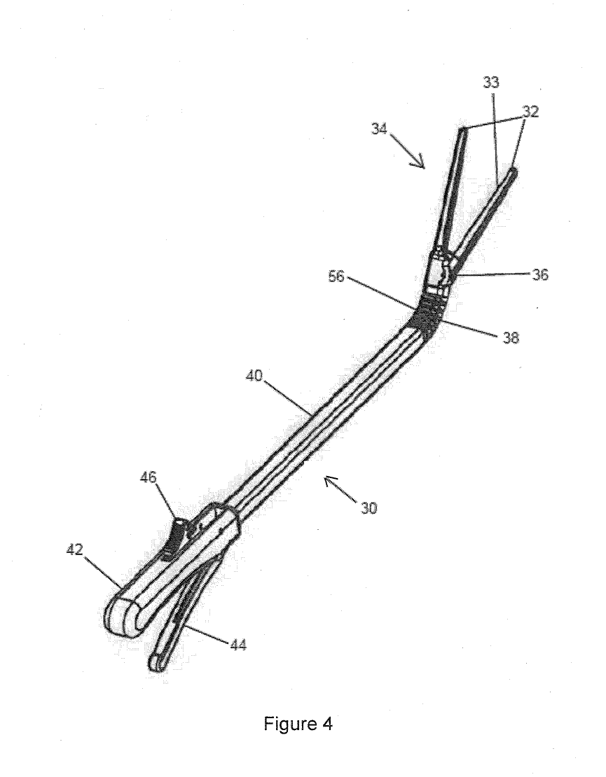

As shown in FIG. 2a, FIG. 2b, FIG. 3 and FIG. 4, jaws 32 may be displaced in a vertical direction by manipulating cable 50 with thumb slide 46. Pulling back on thumb slide 46 exerts a force on gooseneck 38 by way of cable 50 which causes gooseneck 38 to bend in a vertical direction generally orthogonal to the plane defined by the opening and closing of to jaws 32 by compressing gaps 54 between neck segments 48. When a force is no longer exerted on thumb slide 46, a memory of the material of gooseneck 38 returns gooseneck 38 and jaws 32 to a relaxed position. In alternative embodiment, cable 50 may have a spring constant which may provide force returning gooseneck 38 and jaws 32 to the relaxed position. Alternative embodiments may utilize a variety of other actuating mechanisms known in the art to actuate gooseneck 38. In an embodiment, gaps 54 may be insert molded or otherwise filled with materials such as foam or soft rubber in order to aid returning gooseneck 38 to the relaxed position, as well as to reduce a likelihood of pinching patient tissue in gaps 54. In a further embodiment, a thin tubular sheath 56 (FIG. 4) is positioned over gooseneck 38 to protect patient tissue from being pinched in neck segments 48.

In an embodiment, notches in thumb slide 46 allow the gooseneck to be locked at various increments. In an embodiment, the increments are ten (10) degree increments from zero (0) degrees to ninety (90) degrees. In alternative embodiments, increments may be adjustable based on performance needs of a medical professional utilizing ablation device 30. In an embodiment the range of articulation is from zero (0) degrees to sixty (60) degrees with increments of ten (10) degrees. In alternative embodiments, increments may be as small as one (1) degree or less and as large as any value up to ninety (90) degrees. The number of increments may similarly range from one increment to dozens of increments.

In various embodiments, gooseneck 38 is from one (1.0) inch (2.5 centimeters) to two and one-half (2.5) inches (6.4 centimeters) in length. In such varying embodiments, gooseneck 38 having a relatively greater length provides gooseneck 38 with relatively greater ability to articulate. Gooseneck 38 having a relatively shorter length provides gooseneck 38 with relatively less ability to articulate. In an embodiment in which gooseneck 38 has a range of articulation from zero (0) degrees to sixty (60) degrees, gooseneck 38 is approximately one and one-half (1.5) inches (3.8 centimeters) in length.

FIG. 4 shows ablation device 30 with gooseneck 38 articulated at an angle of approximately sixty (60) degrees. As illustrated, gooseneck 38 is contained within tubular sheath 56. As illustrated, tubular sheath 56 is translucent and is selected from biocompatible plastics or rubber materials well known in the art. In alternative embodiments, tubular sheath 56 is opaque and is selected from various biocompatible materials well known in the art. As illustrated, tubular sheath 56 conforms closely with gooseneck 38 in order to reduce cross-sectional form factor to aid use of ablation clamp 30. In such embodiments, tubular sheath 56 extends modestly into gaps 54 in order to provide flexibility of tubular sheath 56. In alternative embodiments, tubular sheath 56 may project some distance from gooseneck 38 and may not extend inside of gaps 54.

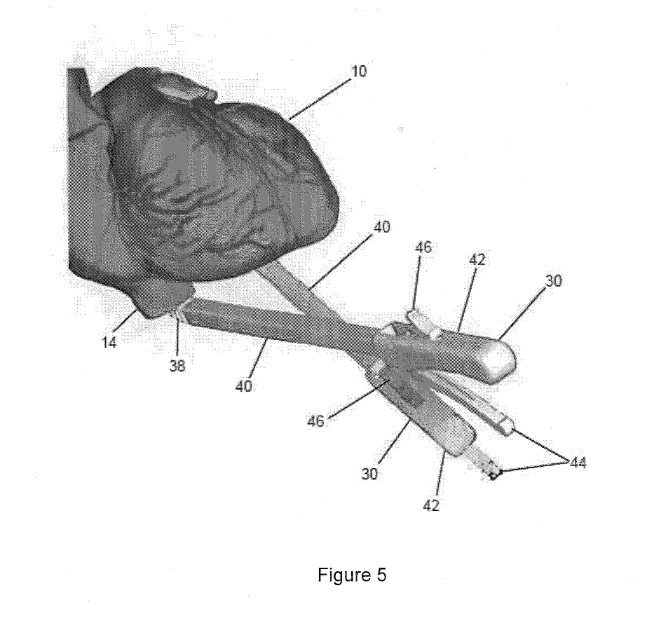

FIG. 5 is an illustration of a pair of ablation clamps 30 being utilized to ablate right pulmonary veins 16 and left pulmonary veins 20 (obscured). As illustrated, while ablation clamp 30 which is utilized to ablate right pulmonary veins 16 approaches from generally directly below right pulmonary veins 16, ablation clamp 30 which is utilized to ablate left pulmonary veins 20 approaches from an angle relative to a vertical axis of heart 10. In an alternative embodiment, utilizing only one ablation clamp 30, right pulmonary veins 16 and left pulmonary veins 20 are ablated serially, in varying embodiments first right pulmonary vein 16 being ablated followed by left pulmonary vein 20, and in alternative embodiments vice versa.

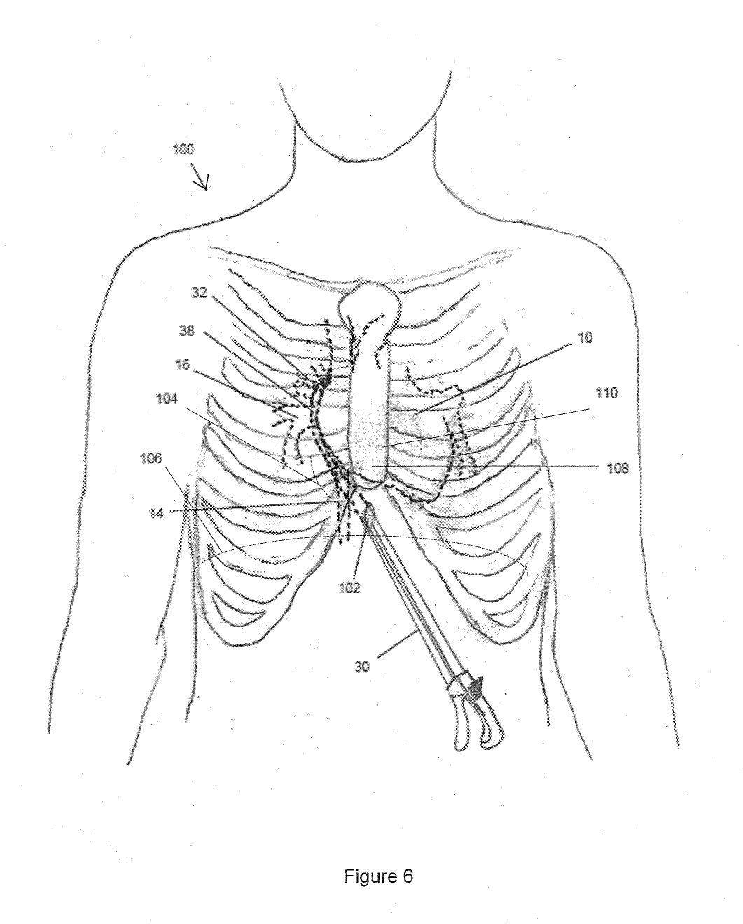

FIG. 6 is an expanded view of ablation clamp 30 in use in heart 10 of patient 100. Preparatory to insertion of ablation clamp 30 in patient 100, substernal incision 102 is created in patient 100. Pericardium 104 is cut near diaphragm 106. Subxiphoid process 108 is cut near sternum 110. Once access is provided to heart 10 ablation, clamp 30 may be inserted for sub-xiphoid use. The most direct path created by substernal incision 102, pericardium 104 and subxiphoid process 108 results in inferior vena cava 14 being generally obstructive of access to right pulmonary veins 16. As illustrated, articulation of gooseneck 38 curves jaws 32 around inferior vena cava 14 places jaws 32 in contact with right pulmonary veins 16. In an embodiment, pericardial reflection 18 between right pulmonary veins 16 and inferior vena cava 14 may be dissected in order to provide access to right pulmonary veins 16. In certain patients, additional dissection of pericardial reflection 18 proximate right pulmonary veins 16 and left pulmonary veins 20 may similarly provide access to right pulmonary veins 16 and left pulmonary veins 20.

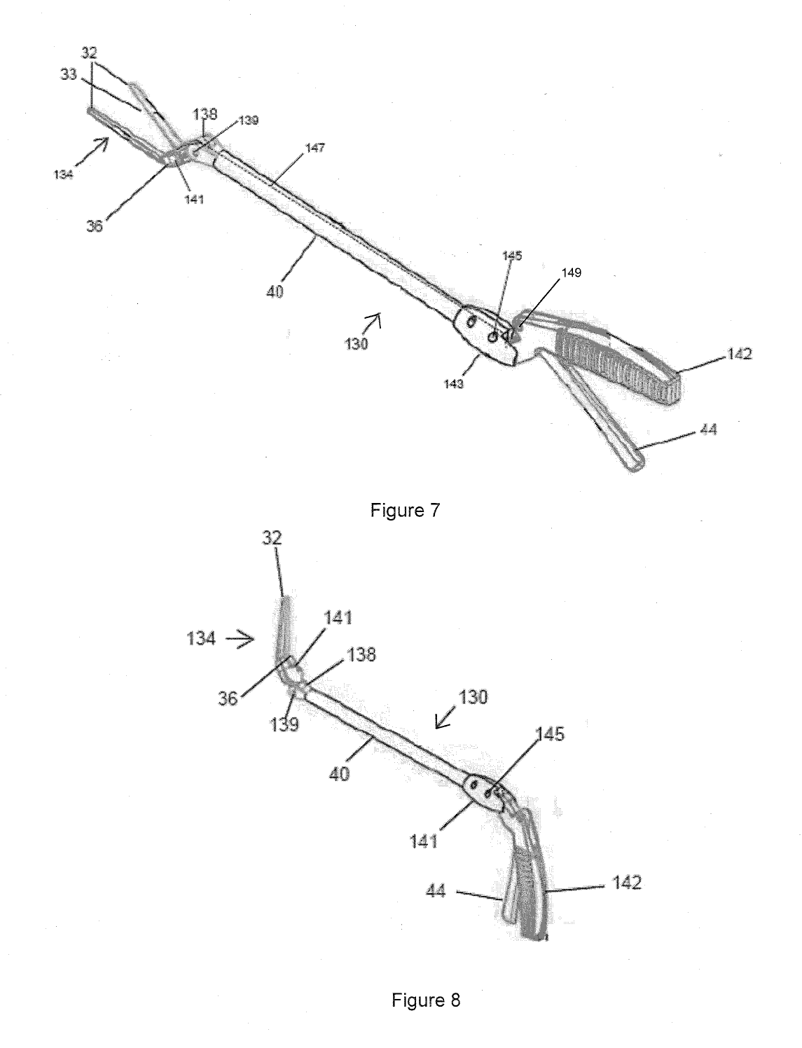

FIG. 7 illustrates an alternative embodiment of sub-xiphoid bipolar ablation clamp 130 having articulating jaws 32 utilizing pivot knuckle 138 for an actuable joint and articulation pivot 145 of neck pivot segment 143 coupled between shaft 140 and handle 142. Pivot knuckle 138 incorporates distal pivot 139, in an embodiment, a pin pivot. Jaw pivot section 141 of jaw segment 134 is coupled to pivot knuckle 138 at distal pivot 139.

Cable 147 (obscured) is coupled to handle 142 at articulation pivot 145 and extends along shaft 140 to distal pivot 139. Cable 147 couples to jaw pivot section 141 of jaw segment 134. By rotating handle 142 about articulation pivot 145 relative to shaft 140, cable 147 acts on jaw pivot section 141, rotating jaw pivot section 141 and, by extension, all of jaw segment 134, relative to shaft 140 about distal pivot 139. In various embodiments, cable 147 completes approximately one full loop about both distal pivot 139 and articulation pivot 145. In alternative embodiments, cable 147 does not complete a full loop but rather extends one length of between distal pivot 139 and articulation pivot 145 in order to connect jaw segment 134 to handle 142.

In an embodiment, a downward articulation of handle 142 relative to shaft 140 results in a similar upward articulation of jaw segment 134 relative to handle 140 due to the force exerted on jaw segment 134 by cable 147. In an embodiment, handle 142 and jaw segment 134 each articulate over an arc of approximately zero (0) degrees to one hundred twenty (120) degrees. In alternative embodiments, the articulation is from approximately zero (0) degrees to seventy-five (75) degrees up to zero (0) degrees to approximately one hundred fifty (150 degrees. In various embodiments, handle is from approximately three (3) inches (7.62 centimeters) to six (6) inches (15.24 centimeters) in length. In an embodiment, handle is approximately five (5) inches (12.70 centimeters) in length. In such an embodiment, trigger pivot 149, about which trigger 44 pivots, has a separation from articulation pivot 145 of approximately One (1) inch (2.54 centimeters) when articulation is zero (0) degrees.

As illustrated, jaws 32 form an angle relative to jaw pivot section 141. As illustrated, when articulation is zero (0) degrees jaws 32 are approximately co-axial with shaft 140 but are offset relative to shaft 140. In various embodiments, a plane of jaws 32 is at a fixed angle with respect to jaw pivot section 141. In such embodiments, the angle between the plane of jaws 32 and jaw pivot section 141 is from thirty (30) degrees to seventy-five (75) degrees. In an embodiment, the angle between the plane of jaws 32 and jaw pivot section 141 is sixty (60) degrees. In such an embodiment, because the plane of jaws 32 are approximately co-axial with shaft 140 at zero (0) degrees articulation, shaft 140 necessarily forms a sixty (60) degree angle with respect to jaw pivot section 141 at zero (0) degree articulation. In alternative embodiments, the plane of jaws 32 is not co-axial with shaft 140 and the angle between jaw pivot section 141 and shaft 140 at zero (0) degrees articulation may vary from the angle between the plane of jaws 32 and jaw pivot section 141. In alternative embodiments, jaws 32 may articulate with respect to jaw pivot section 141.

As illustrated, jaws 32 and ablation elements 33 are utilized from ablation clamp 30. In alternative embodiments, jaws 32 are from approximately two (2) inches (5.08 centimeters) to approximately four (4) inches (10.16 centimeters) in length. In an embodiment, jaws 32 are approximately 2.5 inches (6.35 centimeters) long. In such an embodiment, ablation elements 33 may be from approximately 1.8 inches (4.57 centimeters) to approximately 3.8 inches (9.65 centimeters) long and approximately 0.1 inches (0.254 centimeters) wide. In an embodiment, ablation elements 33 are approximately 2.3 inches (5.84 centimeters) long and approximately 0.1 inches (0.254 centimeters) wide.

In an embodiment, shaft 140 has a circular cross-section having a diameter of approximately 0.5 inches (1.27 centimeters). In alternative embodiments, different cross-sections and different diameters of circular cross-sections may be utilized. In an embodiment, shaft 40 of ablation clamp 30 is utilized. In various embodiments, shaft 140 has a length of from eight (8) inches (20.32 centimeters) to sixteen (16) inches (40.64 centimeters) from neck pivot segment 143 to pivot knuckle 138. In an embodiment, shaft 140 is approximately twelve (12) inches (30.48 centimeters) long.

In various embodiments, pivot knuckle 138 is approximately 0.5 inches (1.27 centimeters) to approximately one (1) inch (2.54 centimeters) long from shaft 140. In an embodiment, pivot knuckle 138 is approximately 0.8 inches (2.03 centimeters) long. In various embodiments, jaw pivot segment 141 is from approximately one (1) inches (2.54 centimeters) to approximately two (2) inches (5.08 centimeters) in length. In an embodiment, jaw pivot segment 141 is approximately 1.3 inches (3.30 centimeters) long.

Similarly with ablation clamp 30, trigger 44 of ablation claim 130 opens and closes jaws 32. In an embodiment, trigger 44 acts only to open and close jaws 32, while ablation energy may be delivered to ablation elements 33 through the use of a separate trigger (not pictured). Alternatively, trigger 44 may provide both opening and closing action for jaws 32 and deliver ablation energy to ablation elements 33 in two stages, as described above with respect to ablation clamp 30.

FIG. 8 illustrates an embodiment of ablation claim 130 in which handle 142 has been articulated down with respect to shaft 140, causing jaw segment 134 to articulate upwards with respect to shaft 140. As illustrated, handle 142 has been articulated downward approximately ninety (90) degrees relative to shaft 140, causing a concurrent ninety (90) degree upward articulation of jaw segment 134 relative to shaft 140.

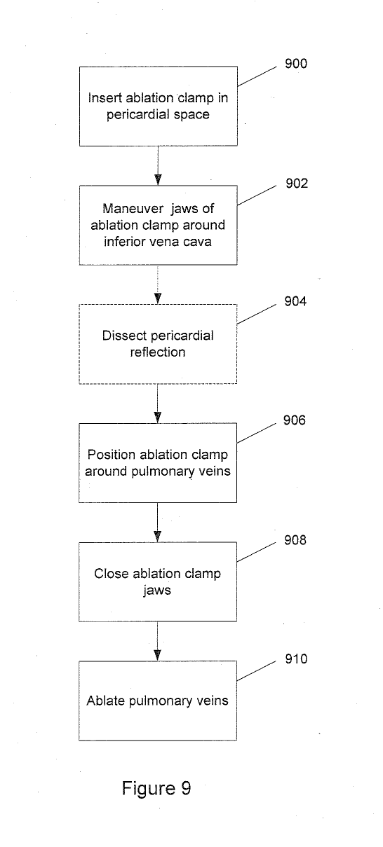

FIG. 9 is a flowchart of a method for ablating right pulmonary veins 16 using ablation clamp 30. Jaws 32 of ablation clamp 30 are inserted (900) into the pericardial space of the patient proximate heart 10. Jaws 32 are maneuvered (902) around inferior vena cava 14, so that both jaws 32 pass to one lateral side of inferior vena cava 14. In various embodiments, pericardial reflection 18 between inferior vena cava 14 and right pulmonary veins 16 is dissected (904) to permit access of one of jaws 32 to one lateral side of right pulmonary veins 16 while the other of jaws 32 passes to the opposite lateral side of right pulmonary veins 16.

Jaws 32 of ablation clamp 30 are positioned (906) proximate right pulmonary veins 16 by articulating gooseneck 38. As positioned, one jaw 32 may be on one lateral side of right pulmonary veins 16 and the other jaw 32 on the opposing lateral side of right pulmonary veins 16. Jaws 32 are clamped (908) using trigger 44, bringing ablation elements 33 into contact with right pulmonary veins 16. Ablation energy is delivered (910) to right pulmonary veins 16 in order to create the lesion.

In an alternative embodiment, ablation clamp 130 is utilized according to the above steps. In alternative embodiments, left pulmonary veins 20 may be ablated by generally repeating the steps of FIG. 9. However, ablation clamp 30 would not be maneuvered with respect to inferior vena cava 14, as in step (904), but would rather approach left pulmonary veins 20 directly. Pericardial reflection 18 between superior vena cava 12 and right pulmonary veins 16 and left pulmonary veins 20 would optionally be dissected (904) and jaws 32 positioned (906) and clamped (908) around left pulmonary veins 20.

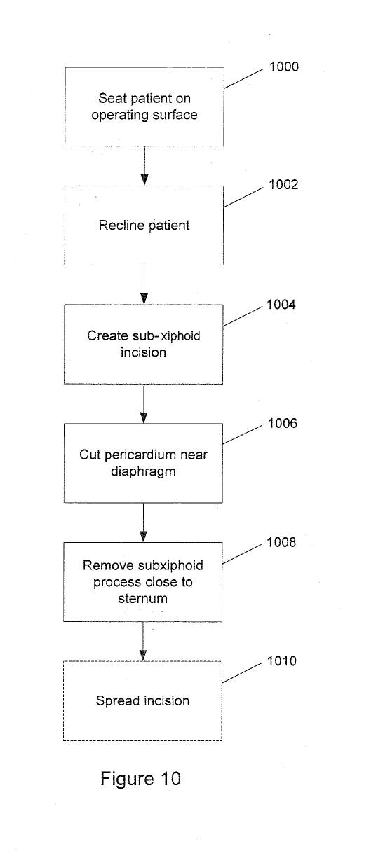

FIG. 10 is a flowchart for a procedure which may be preparatory to implementing the ablation method of FIG. 9. A patient is seated (1000) on an operating surface. In various embodiments, the operating surface is part of a reclining table, examples of which are well known in the art. The operating surface, and by extension the patient, is reclined (1002) from between approximately ten (10) degrees and approximately thirty-five (35) degrees. In various embodiments the degree of recline is selected in order to give a medical professional a preferred sub-xiphoid angle of approach to heart 10. In an embodiment, the patient is reclined approximately twenty (20) degrees.

Sub-xiphoid incision 102 is created (1004) in the skin of the patient. In various embodiments initial sub-xiphoid incision 102 is wide enough to permit introduction of jaws 32 and a portion of shaft 40 proximate heart 10. In various of such embodiments, jaws 32 are in open position, while in other embodiments jaws 32 are in a closed position. In alternative embodiments, sub-xiphoid incision 102 is not initially large enough to permit introduction of 32 and shaft 40, and is instead large enough to allow the introduction of cutting devices. In various embodiments, sub-xiphoid incision 102 is from approximately 1.0 centimeters in length to approximately 12.0 centimeters in length. In such circumstances the length of incision 102 may vary dependent on factors including anatomical features of the patient, visualization devices utilized during use of ablation clamp 30 and the relative skill of the medical professionals conducting utilizing ablation clamp 30. In an embodiment sub-xiphoid incision 102 is approximately three (3) inches (7.62 centimeters) long.

After creation of sub-xiphoid incision 102, the pericardium 104 of heart 10 is cut (1006) proximate the diaphragm 106 of the patient to create access to heart 10. Similarly with sub-xiphoid incision 102, the cut in pericardium 104 may be wide enough to permit passage through the cut of jaws 32 and a portion of shaft 40. As with the creation of sub-xiphoid incision 102, in various embodiments the pericardial cut is large enough to allow jaws 32 to pass through in an open position in some embodiments and in a closed position in other embodiments. Subxiphoid process 108 of the patient is then removed (1008) proximate sternum 110 to create a gap. In an embodiment, subxiphoid process 108 is removed as close to sternum 110 as may be safely attained. In alternative embodiments, subxiphoid process 108 is removed somewhat farther away from sternum 110, albeit still close to sternum 110. Once steps (1006) and (1008) have been performed, sub-xiphoid incision 102 may be spread (1010) if necessary to permit introduction (FIG. 9, 900) of jaws 32 and shaft 40.

Thus, embodiments of the invention are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A sub-xiphoid ablation clamp for ablating tissue of a patient, comprising:
an elongate shaft having a major axis, a proximal end and a distal end;
first and second opposing jaws configured to open and close along a first plane, said first and second opposing jaws having a first and second ablation element positioned along said first and second jaws, respectively, configured to ablate said tissue positioned therebetween;
an actuable joint operatively coupled between said distal end of said elongate shaft and said first and second opposing jaws, said actuable joint being configured to move said opposing jaws to a selectable angle relative to said major axis of said elongate shaft along a second plane orthogonal to said first plane of said opposing jaws;
a handle operatively coupled to said proximal end of said elongate shaft, comprising:
an actuator operatively coupled to said actuable joint and configured to actuate said actuable joint; and
a trigger mechanism operatively coupled to said first and second opposing jaws, said trigger mechanism being operable to open and close said opposing jaws.

2. The sub-xiphoid ablation clamp of claim 1 wherein said actuable joint is comprised of a plurality of articulated segments.

3. The sub-xiphoid ablation clamp of claim 2 wherein said actuable joint is a gooseneck.

4. The sub-xiphoid ablation clamp of claim 1 wherein said actuable joint is configured to move said operable jaws along said second plane with respect to said major axis of said shaft only in a first direction.

5. The sub-xiphoid ablation clamp of claim 1 wherein said actuable joint comprises a pivot joint.

6. The sub-xiphoid ablation clamp of claim 5 wherein said actuator comprises an actuator pivot operatively coupling said handle to said shaft and wherein a movement of said handle relative to said shaft causes a movement of said first and second opposing jaws relative to said shaft about said pivot joint.

7. The sub-xiphoid ablation clamp of claim 6 wherein moving said handle a distance in a first direction first and second opposing jaws said distance in a second direction.

8. The sub-xiphoid ablation clamp of claim 1 wherein said actuator is configured to actuate said actuable joint to a plurality of predetermined angles relative to said major axis of said shaft.

9. The sub-xiphoid ablation clamp of claim 8 wherein said plurality of predetermined angles are at predetermined increments.

10. The sub-xiphoid ablation clamp of claim 9 wherein said predetermined increments are approximately ten degrees.

11. A method of sub-xiphoid ablation of a vein of a heart of a patient with a sub-xiphoid ablation clamp comprising an elongate shaft having a major axis, first and second opposing jaws configured to open and close along a first plane, said first and second opposing jaws comprising a first and second ablation element, an actuable joint operatively being configured to move said opposing jaws to a selectable angle relative to said major axis of said elongate shaft along a second plane orthogonal to said first plane of said opposing jaws, comprising the steps of:
inserting said ablation clamp within said patient from a sub-xiphoid direction;
positioning said opposing jaws proximate said vein;
moving said opposing jaws along said second plane to a particular selectable angle with respect to said major axis of said shaft position said vein between said opposing jaws;
clamping said vein between said opposing jaws by closing said opposing jaws along said first plane; and
delivering ablation energy to said vein from said first and second opposing electrodes.

12. The method of claim 11 wherein said vein is a right pulmonary vein.

13. The method of claim 12, further comprising the step, before said inserting step, of creating an incision in skin of the patient below a sternum of said patient, and wherein said inserting step comprising inserting said sub-xiphoid ablation clamp into said incision.

14. The method of claim 13, further comprising the steps, after said creating an incision step, of:
creating an incision in a pericardium of said heart of said patient;
creating a gap in said subxiphoid process; and
passing said jaws of said sub-xiphoid ablation clamp though said incision in said pericardium and said gap in said subxiphoid process.

15. The method of claim 14 wherein said gap in said subxiphoid process is created by removing a portion of said subxiphoid process proximate a sternum of said patient.
